(19) United States
(12) Patent Application Publication
(10) Pub. No.: US 2002/0192747 A1
(43) Pub. Date: Dec. 19, 2002

(54) Expression and purification of galactose oxidase

(76) Inventors: James W. Whittaker, Hillsboro, OR (US); Mei M. Whittaker, Hillsboro, OR (US)

Correspondence Address: Howard M. Eisenberg, Esq., Suite 1600, 601 S.W. Second Avenue, Portland, OR, 97204, US

(21) Appl. No.: 09/866,060

(22) Filed: May 25, 2001

Related U.S. Application Data
(60) Provisional application No. 60/207,846, filed on May 30, 2000.

(51) Int. Cl. C12P 21/02; C12N 9/02; C12N 1/18; C12N 15/74

(52) U.S. Cl. 435/69.1; 435/254.2; 435/320.1; 435/254.23; 435/189

(57) ABSTRACT

A method for the production and purification of galactose oxidase in which a yeast is transformed with a vector coding for a fusion protein of a signal peptide and galactose oxidase and an inducible promoter that promotes the production of the signal peptide, the yeast is induced to produce the fusion protein, the galactose oxidase is removed from the fusion protein while it is in the yeast cell, and the galactose oxidase is secreted from the yeast cell.

This application claims the benefit of the filing date of pending Provisional Application No. 60/207,846, filed on May 30, 2000, which application is incorporated herein by reference.

The invention was developed under a grant from the National Institute of Health, grant number GM 46749.

### FIELD OF THE INVENTION

The invention pertains to the field of recombinantly expressed proteins.

### BACKGROUND OF THE INVENTION

Galactose oxidase is a fungal copper metalloenzyme secreted by the filamentous fungus Dactylium dendroides. Galactose oxidase is a fungal radical copper oxidase that catalyzes the oxidation of a variety of primary alcohols (RCH₂OH) to the corresponding aldehyde, coupling this reaction to the reduction of dioxygen to hydrogen peroxide:

RCH₂OH+O₂→RCHO+H₂O₂.

Galactose oxidase oxidizes only primary alcohols, and as a result of this strict regioselectivity secondary alcohols do not serve as substrates. Among hexoses there is a remarkably high degree of specificity for galactose and its derivatives, making galactose oxidase important for biomedical applications, including clinical assays for galactose in blood and other biological fluids, histochemical studies, and recently, in early detection of cancer. The enzyme is also of interest for applications requiring in situ generation of hydrogen peroxide and in preparation of bioconjugates.

This enzyme is widely used in clinical diagnostic tests and histochemical analyses, making it a biotechnologically significant enzyme. In biomedical applications, it is utilized in the quantitative analysis of galactose in blood and other biological samples. It is also useful in the identification and characterization of glycoproteins in tissues, which can be employed to detect certain types of cancer and other pathologies. In basic research, galactose oxidase is a reagent that can modify cell surfaces for cell biology applications and generate hydrogen peroxide for a variety of experimental processes.

Expression from D. dendroides is not very efficient, yielding approximately 80 mg enzyme for 45 liters of medium (2 mg/L) after one week of growth. At present, galactose oxidase is commercially available, for example from Sigma (Sigma-Aldrich Co., St. Louis, Mo.), for $63/450 units as a partially purified enzyme preparation. With the specific activity ranging from 500-1500 units/mg, the product is rather costly (50-150 dollars/mg) and may cause many problems due to this variability. The fungus secretes biopolymers and other enzymes that complicate the isolation of pure galactose oxidase. For this reason Sigma states that their product contains trace amounts of catalase (which interferes with reactions depending on generation of hydrogen peroxide) and may contain other enzymatic activities. In addition, galactose oxidase must be activated by an oxidizing agent to reach maximum catalytic activity. Impurities in its preparation have been shown to inhibit the activation process, dramatically reducing the expected activity.

The relatively low level of secretory expression of galactose oxidase by Dactylium dendroides has motivated interest in developing alternative expression systems for this enzyme. Previous studies have explored Aspergillus niger as an expression host, providing higher enzyme yields but retaining many of the difficulties of filamentous fungus fermentation. For example, the purity of the recombinant galactose oxidase produced by this method is not significantly better than that of the native enzyme because the host fungus also produces large number of contaminating secretory products.

### SUMMARY OF THE INVENTION

In one embodiment, the invention is a new method that has been developed for the production of galactose oxidase in yeast. The method of the invention results in a high level of the recombinant enzyme produced and allows the protein to be efficiently secreted and processed to produce a homogeneous population of mature enzyme in the extracellular media. This system permits high density fermentation, tightly regulated expression, and efficient secretion of recombinant proteins, with yields in some instances exceeding 1 g/L.

The method utilizes an inducible promoter and a leader peptide that enables an efficient expression and facile purification scheme that allows the isolation of galactose oxidase that is purer, more active, and more economical than that which is presently commercially available.

In another embodiment, the method is a purification scheme for isolation of galactose oxidase. This method allows the isolation of galactose oxidase that is purer, more active, and more economical than that which is presently commercially available.

In the specification that follows, the invention is illustrated using a particular yeast, Pichia pastoris, and a particular expression system utilizing a methanol-induced promoter. The description is intended to be illustrative of the invention. One skilled in the art will understand that the invention may be practiced with other yeasts, such as Hansenula spp. and Saccharomyces spp. Likewise, one skilled in the art will understand that other expression systems, including those that utilize promoters that are induced by other than methanol and including those that use leader peptides other than the Aspergillus gla leader peptide illustrated below, are suitable for the invention.

### BRIEF DESCRIPTION OF THE DRAWINGS

FIG. 1 shows a representation of the pPICZBgla expression vector.

FIG. 2 shows a representation of the pPICZBglaGAOX expression vector.

### DETAILED DESCRIPTION OF THE INVENTION

The invention is illustrated with a preferred vector and a preferred yeast, Pichia pastoris. The invention permits high-level expression of recombinant galactose oxidase in a yeast. Pichia pastoris is preferred as it is widely used for heterologous protein expression, secretes few proteins, and contains no endogenous galactose oxidase.

Using published DNA sequences, we isolated the galactose oxidase gene from a D. dendroides cDNA library. The cDNA encoding the mature galactose oxidase protein was fused to the DNA coding for the Aspergillus glucoamylase leader peptide and was inserted downstream of a methanol-inducible promoter in the pPICZ vector (Invitrogen, San Diego, Calif.). P. pastoris cells transformed with this expression plasmid were grown to a high cell density and then shifted to methanol growth medium to trigger large-scale production of the fusion protein.

The glucoamylase leader directs the fusion protein to be secreted into the media but is preferably removed (processed) before the mature galactose oxidase leaves the cell. Use of other leader peptides, such as the native galactose oxidase leader peptide which is commonly employed for secretion, resulted in less efficient processing of the fusion protein and a less homogenous population of secreted galactose oxidase. For this reason, this system is not preferred.

The preferred system, as illustrated herein, provides for methanol induction and efficient secretion of a homogenous mature enzyme to a level of at least 200 mg/5 liters of culture (40 mg/L) after one day of methanol culturing or 2.5 grams/5 liters after three days. We have also developed an additional process for purification and activation of the product to a form exhibiting a uniformly high specific activity of 2500 units/mg. This procedure consists of oxidation using an oxidant, preferably an inorganic oxidant, which is preferably ferricyanide (hexacyanoferrate(III), K₃Fe(CN)₆). Other suitable oxidants include molybdicyanide and iridium(IV) chloride. The procedure also includes size exclusion chromatography and dialysis, and allows the enzyme to metabolize any impurities and reach full activation.

The physical characteristics (molecular weight, Kₘ, etc.) of the P. pastoris derived galactose oxidase are identical to those of the native enzyme. In summary, our system is superior to traditional methods for the preparation of galactose oxidase because of the high level of expression of homogeneous enzyme along with its efficient isolation and activation.

The invention is further described in the following non-limiting examples.

### EXAMPLE 1

Isolation of GAOX cDNA.

D. dendroides (ATCC 46032, identical to the Fusarium spp. strain NRRL 2903) was grown on sorbose medium for 3 days at room temperature in the dark for maximal expression of galactose oxidase (GAOX). Expression of GOAX was verified experimentally by assaying the culture media. The culture was filtered through autoclaved cheesecloth to collect the fungal mycelia which were briefly dried on sterile filter paper. Total RNA was isolated from 100 mg of frozen mycelia triturated in liquid nitrogen using the QIAGEN RNeasy Plant Mini Kit (Auiagen, Madison, Wis.). Poly-A⁺ mRNA was isolated from total RNA using the QIAGEN Oligotex mRNA spin column protocol. First-strand cDNA product prepared using the Stratagene RT-PCR kit (Stratagene, San Diego, Calif.) was used to synthesize GAOX cDNA with primers based on the published sequence (GenBank accession number 167225):

5′-CGCTTCGAATGAAACACCTTTTAACACTCGCTCTTTGCTTCAGCAGC-3′
5′-CGCTCTAGATCACTGAGTAACGCGAATCGTCGAAGCCACACTAGGAACACCG-3′

An alternative forward primer was used to synthesize truncated GAOX cDNA coding for the mature protein with a 5′ BssHII restriction site:

5′-CGTGCGCGCCTCAGCACCTATCGGAAGCGCCATTTCTCGCAACAACTG-3′.

### EXAMPLE 2

Vector Construction.

An 87-mer oligonucleotide synthetic minigene was designed based on the Aspergillus niger glaA glucoamylase signal peptide (gla) sequence, modified through silent mutagenesis to incorporate a BstBl restriction site 7 nt upstream from the fusion junction and including ClaI and EcoRI terminal sites for cloning into the pPICZB expression vector (Invitrogen, San Diego, Calif.

```
    ClaI | BstBI | BssHII | EcoRI
ATCGATATGTCGTTCCGATCTCTACTCGCCCTGAGCGGCCTCGTCTGCACAGGGTTGGCAAATGTGATTTCGAAGCGCGCAGAATTC
            Met Ser Phe Arg Ser Leu Leu Ala Leu Ser Gly Leu Val Cys Thr Gly Len Ala Asn Val Ile Ser Lys Arg Ala Glu Phe
                                                                                                                                                                                                        \hat{ }
                                                                                                                                                                                                          Kex2
            | pre | pro |
                                                                              gla signal sequence mature protein
```

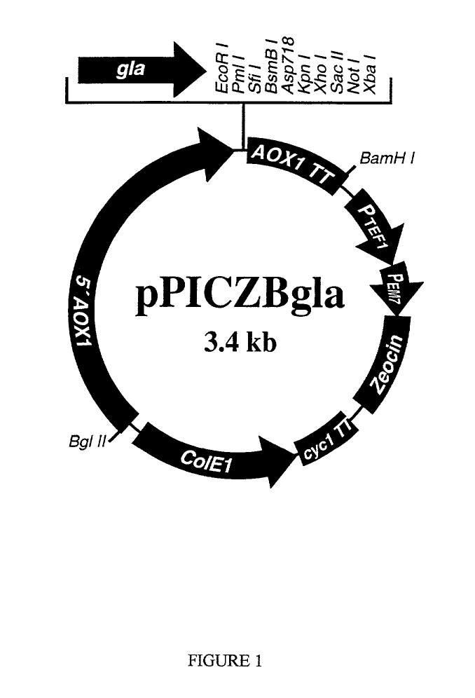

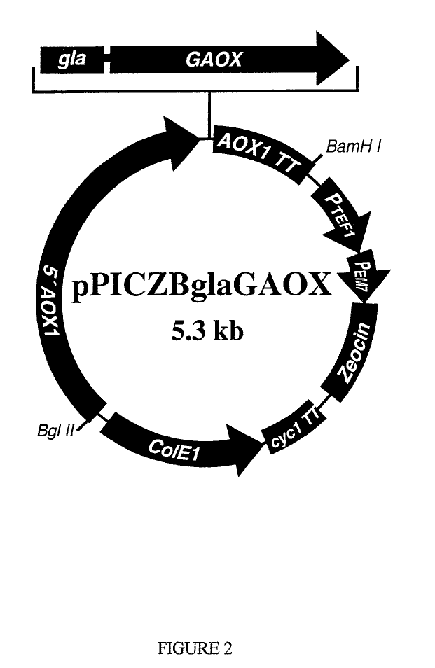

While the BstBI site at −7 does not impose any sequence restriction on the fusion protein, the BssHII site overlaps the boundary of the gla fusion sequence and constrains the first codon of the downstream sequence to GCX, requiring the mature protein product produced by cleavage of the gla prosequence to begin with an N-terminal alanine residue. Elimination of any BssHII sites in the flanking region of the vector is necessary in order to make use of the BssHII site in the gla signal sequence for construction of gene fusion products. For this reason, the unique BssHII site within the ble marker of the pPICZB vector was removed by silent mutagenesis using Quickchange mutagenesis procedure and the altered ble* coding sequence in the BssHII-resistant product was confirmed by DNA sequence analysis. Ligation of BstBI/EcoRl digested ble* mutant derivative of the pPICZB plasmid with ClaI/EcoRl digested gla minigene generated the pPICZBgla vector, as shown in FIG. 1. The BssHII/XbaI digested pPICZBgla vector was ligated with similarly digested truncated GAOX PCR product to generate the expression vector pPICZBglaGAOX, as shown in FIG. 2, coding for a fusion protein combining the A. niger gla signal peptide with the mature GAOX polypeptide. For comparison, the full length GAOX cDNA (including the native pre-pro signal sequence) was also cloned into pPICZB to generate the expression vector pPICZBGAOX (not shown).

### EXAMPLE 3

Transformation of Pichia pastoris

Pichia pastoris X33 was transformed with PmeI-linearized plasmid by electroporation. Multicopy integrants were selected with 1 mg/ml Zeocin and screened for high level methanol-induced expression of GAOX. Large scale high density fermentation of the Pichia pastoris expression strains was performed as previously described with slight modifications. An overnight culture of P. pastoris (300 mL) was used to inoculate 5 liters of FM22 medium supplemented with PMT4 trace metals solution in a BioFlo 3000 Bioreactor equipped with a 14 L fermentation vessel. The initial glycerol batch cultivation was run for approximately 24 hours with vigorous agitation (500 rpm) and air/oxygen gas delivery regulated to maintain 40% dissolved oxygen level. The pH of the medium was maintained at 4.5 during this phase by the automatic addition of 5 N NH₄OH, and 5% KFO™ 673 antifoam (Kabo Chemical Co., Inc., Jackson Hole, Wyo.) in methanol was delivered as required. Temperature was maintained at 29° C. using a NESLAB refrigerated circulator (CFT-33) in the external cooling loop. When the initial quantity of glycerol was consumed, the batch feeding phase was initiated with delivery of 400 mL of 50% glycerol at 15 mL/L·h. When the addition was complete, a solution of filter-sterilized 10% casamino acids in 0.5 M KHPO₄ buffer pH6 (50 g casamino acids/500 mL KHPO₄ buffer) was added to the medium and the pH was raised to 6.0 by addition of 5 N NH₄OH. During the induction phase, the temperature was lowered to 25° C. and methanol feeding was initiated using a Watson-Marlow peristaltic pump for continuous-duty delivery of methanol (containing 4 mL of PMT4 trace metals solution and 12.5 mL of 40 mM CUSO₄ solution per liter of methanol). Methanol addition was maintained constant at a rate of ˜15 mL/h for 3 days.

### EXAMPLE 4

Purification of GAOX

Pichia cell biomass was removed from the fermentation broth by centrifugation and the supernatant was passed through a Millipore Opticap filter unit with 0.5 μm nominal pore size low protein binding Milligard media to remove residual particulates. The filtrate was concentrated to about 100 mL through a Millipore Pellicon 2 cassette filter unit (0.3 m²) with PL-series regenerated cellulose membranes (PLGC 10K). The concentrated filtrate was dialyzed for 12 h against 4 L of 20 mM KHPO₄ buffer (pH 7). After centrifugation, the dialysate was loaded onto a Whatman DE-52 anion exchange chromatography column (5×50 cm) equilibrated and developed in the same buffer. The pale blue (inactive) GAOX passed through the column and was recovered from the flow-through fractions free from intense green contaminants which were retained on the column.

Unlike GAOX isolated from D. dendroides, which is a mixture of inactive blue enzyme and active green enzyme, the GAOX isolated from yeast, such as Pichia, fermentation is completely inactive (blue) as a consequence of the presence of methanol in the culture medium. Inactive blue GAOX can be activated by treatment with oxidant (for example, hexacyanoferrate(III) (K₃Fe(CN)₆). Thus, one-tenth volume of 0.5 M K₃Fe(CN)₆ was added to the enzyme (20 mg/mL in 20 mM KHPO₄, pH 7) and immediately loaded onto a BioGel P-30 size exclusion chromatography column (bed volume 10× the sample volume) and the active green enzyme fractions pooled and dialyzed against KHPO₄ buffer or water for 12 h. During this time the enzyme consumes substrate-like contaminants (e.g., protein glycoconjugates) and the H₂O₂ product is removed by dialysis. Under limiting oxygen (e.g., in an unstirred sample) the active enzyme may become colorless through reduction, a process that is reversible by addition of air. The highest activity GAOX is obtained by repeating the K₃Fe(CN)₆ treatment with desalting but without dialysis.

### EXAMPLE 5

Enzyme Assays

Galactose oxidase activity was measured by oxygen uptake in a Clark oxygen electrode. The assay contained 50 mM O-methyl α-D-galactopyranoside, with or without 2 mM K₃Fe(CN)₆ in 50 mM KHPO₄ (pH 7). The electrode was calibrated using the protocatechuic acid/protocatechuate dioxygenase reaction. Alternatively, the direct assay based on oxidation of 3-methoxy benzyl alcohol may be used for determining galactose oxidase activity.

Transformation of a yeast, such as Pichia pastoris, with the linearized pPICZBglaGAOX plasmid under conditions of strong antibiotic selection produces transformants that efficiently secrete galactose oxidase under methanol induction. A single major protein band (68 kDa) is observed by SDS-PAGE analysis of the culture medium, associated with galactose oxidase activity detected by Clark oxygen electrode assay. In contrast, Pichia transformed with the pPICZBGAOX vector produced two major protein products (68 and 70 kDa).

The yield of active, intact GAOX is sensitive to the culture conditions. During fermentation with the illustrative system described in the Examples, it is essential to raise the pH to 6 during the methanol induction period to stabilize the protein. Galactose oxidase is not stable at lower pH for extended period of times typically required for fermentation. In shake flask culture, we have found that a decrease in growth temperature from 30° C. to 25° C. increased the yield of galactose oxidase 3-4 times. In a bioreactor with automatic delivery of methanol, the yield of GAOX increased ten-fold (from 50 mg/L to 0.5 g/L) when the temperature was decreased from 29° C. to 25° C. Using the peroxidase/o-tolidine assay system, this gives 16,000 Worthington units per milligram of protein.

Further modifications, uses, and applications of the invention described herein will be apparent to those skilled in the art. These modifications include the use of yeast other then Pichia, induction systems other than methanol induction, and process variables, such as pH, time, temperature, particular reagents, and concentrations other than illustrated above. It is intended that such modifications be encompassed in the following claims.

1. A method for producing isolated galactose oxidase comprising transforming a yeast with a vector comprising a nucleic acid sequence encoding a fusion protein of a signal peptide and galactose oxidase, and an inducible promoter that regulates transcription of the sequence encoding said fusion protein, culturing said transformed yeast, inducing said promoter to cause yeast to produce said fusion protein, removing within the yeast said signal peptide from the galactose oxidase, and secreting the galactose oxidase from the yeast.

2. The method of claim 1 which further comprises activating the secreted galactose oxidase by treatment with an oxidant.

3. The method of claim 1 wherein the yeast is Pichia spp.

4. The method of claim 1 wherein the signal peptide is an Aspergillus niger gla signal peptide.

5. The method of claim 1 wherein the inducible promoter is a methanol-inducible promoter.

6. A vector for transforming yeast comprising a nucleotide sequence encoding a fusion protein of a signal peptide and galactose oxidase, and an inducible promoter that regulates transcription of the sequence encoding said fusion protein.

7. The vector of claim 6 wherein the yeast is Pichia spp.

8. The vector of claim 6 wherein the signal peptide is an Aspergillus niger gla signal peptide.

9. The vector of claim 6 wherein the inducible promoter is a methanol-inducible promoter.

10. A nucleotide sequence encoding a fusion protein of a signal peptide and galactose oxidase, and an inducible promoter that regulates transcription of the sequence encoding said fusion protein.

11. The nucleotide sequence of claim 10 wherein the signal peptide is an Aspergillus niger gla signal peptide.

12. The vector of claim 10 wherein the inducible promoter is a methanol-inducible promoter.

13. A fusion protein comprising a signal peptide and galactose oxidase, wherein the signal peptide is an Aspergillus niger gla signal peptide.

14. A method for activating galactose oxidase comprising treating the galactose oxidase with an oxidant.

15. The method of claim 14 wherein the oxidant is selected from the group consisting of ferricyanide, molybdocyanide and iridium(IV) chloride.
